(19) United States
(12) Patent Application Publication
(10) Pub. No.: US 2007/0092464 A1
(43) Pub. Date: Apr. 26, 2007

(54) Betadine neutralizer

(76) Inventor: Rocky Joe Duff, Spokane, WA (US)

Correspondence Address: CHRISTOPHER D. HARRINGTON;SUITE 350, 5300 CORPORATE GROVE SE, GRAND RAPIDS, MI, 49512, US

(21) Appl. No.: 11/256,731

(22) Filed: Oct. 25, 2005

(51) Int. Cl. A61K 8/19

(52) U.S. Cl. 424/70.1

(57) ABSTRACT

A new preparation for the removal of iodine stains in clinical or home environments is disclosed as an aqueous solution of a metabisulfite in the preferred range of 0.01 to 0.04 percent by weight, metabisulfite to water. The preferred metabisulfite for this purpose is sodium metabisulfite. In one embodiment of the invention, wipes saturated with the metabisulfite solution are packaged individually for use by a health care professional in removing iodine staining from the skin of a patient, and/or other stained areas.

### CROSS REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

The present invention relates to a compound for cleaning Iodine stains resulting from first aid and surgical treatments. More specifically, the present invention is an aqueous solution of sodium metabisulfite used to neutralize and clean Iodine related stains on skin, fabrics, and any other materials that are subject to Iodine type staining.

Tincture of Iodine has been used for nearly two hundred years, having been discovered by Bernard Courtois in 1811. It took some years thereafter for the characteristics and properties of Iodine to be determined, with the result that it is now known to be a halogen, like chlorine and bromine, although it is the least reactive of the halogens. Over time processes for the production of Iodine were developed and eventually its use as an effective microbicide or antiseptic was discovered and it is perhaps best know by the general public for this purpose. The usual preparation of Iodine in this form is tincture of Iodine which is typically in the range of 2% to 3% elemental Iodine in a solution of water and ethanol. However, elemental Iodine even though it is a weakly reactive halogen, does have a propensity to cause skin irritations including burning if it is left on the skin in high enough concentrations.

The Purdue Frederick Company developed an Iodine based preparation that is known generally as “Betadine” and this solution (in different forms) is widely used in the health care fields and in home and first-aid applications as well. Betadine is a water soluble polymeric compound that is able to carry Iodine that is dissociable into elemental Iodine in a given application. The general formula for Betadine is (C₆H₉I₂NO)ₙ·Iₓ and is verbally described as 1-ethyenyl-2-pyrrolidinone homopolmer compound with Iodine, and sometimes known more simply as povidone Iodine. Betadine typically is less of an irritant to humans than tincture of Iodine, although reports indicate that Betadine still can cause irritation to skins and eyes in animals and humans. The de-natured characteristics of Betadine has made it a very popular antiseptic in operating rooms and in emergency rooms, reducing the irritant characteristics of the Iodine solutions to some degree. In actual use though, Betadine, like elemental Iodine, has the propensity to stain skin, fabric, and even floor tiles at times, with a dark burgundy tint that is highly resistant to detergent washing.

The usage of Betadine and Betadine-like solutions are most often indicated in the operating room and emergency room environments. These solutions, sometimes referred to as “scrubs,” are useful in setting up an antiseptic field for whatever invasive technique is to be used on a patient. The area of interest is swabbed with the Betadine scrub and the scrub solution is left on the skin to retard any microbial potentials. Both Betadine and Iodine scrubs have been shown to be remarkably effective in knocking down various microbial populations, including bacteria, viral, and fungal, and leaving the scrub wash on the patient during a procedure is considered to be a prophylactic benefit since it will continue to knock-down the microbial potential during the surgery, and thereafter after the closing of any sutures or incisions. This practice also has the detriment of ensuring that the Iodine staining will be firmly entrenched when the patient is in the recovery room.

Purdue Frederick does market a product specifically directed to the cleaning of Betadine stains; their product going by the trade name of Povi-Clens and which is provided in a spray formulation. This product is based on alkaline detergent chemistry, to the extent its constituents are disclosed by Purdue Frederick; no Material Safety Data Sheet could be located for this product. It cannot be determined if Povi-Clens utilizes any reducing agents at all, but clearly if it did it would not be sodium metabisulfite given the alkaline matrix of the Povi-Clens product. Sodium metabisulfite would lose its effectiveness in an alkaline matrix and would not be reactive as a reducing agent.

Povi-Clens might possibly, as well as other known Iodine cleaning products, use sodium thiosulfate chemistry as a reducing agent. Sodium thiosulfate, with a chemical formula of Na₂S₂O₃, is capable of neutralizing halogens such as chlorine, bromine, and of course, Iodine. Formulating sodium thiosulfate into a detergent type product is more compatible than trying to use sodium metabisulfite since sodium thiosulfate is slightly alkaline in its normal state. Some of the other Iodine remover products, like one from Professional Disposables International (PDI) known as “IO-Gone,” utilize an alcohol matrix along with the aqueous solution of sodium thiosulfate. The IO-Gone product, in particular, uses a 50% solution of isopropyl alcohol with 3% sodium thiosulfate in a water solute. Another well-known Iodine stain remover on the market is the 3M product known merely as “Remover Lotion.” 3M sells this product in conjunction with its version of the polymerized Iodine preparation scrub known as 3M “Duraprep.” Duraprep is essentially the same type of de-natured Iodine chemistry as Betadine and would be expected to react in the same way. The Remover Lotion, however, includes additional chemistry to promote the isopropyl alcohol and sodium thiosulfate matrix in conjunction with certain stearates resulting in a thickened lotion type of solution that is applied to the patient.

Both sodium thiosulfate and sodium metabisulfite have the potential to provoke allergic reactions in a very small number of people, however, with respect to sodium metabisulfite these reactions typically arise from the ingestion of the chemical, while sodium thiosulfate is associated with the potential for topical allergic reactions that can, at times, be serious. In the case of sodium metabisulfite, there are a number of people who demonstrate an allergic reaction to sulfites in general, thus many food preservatives and food preparation materials must be made sulfite-free for such people. The route for provoking a sulfite reaction is typically through oral ingestion, which in the case of a post-surgical cleanser, would not likely be a problem. In either event, it is believed that the usage of either sodium thiosulfate or sodium metabisulfite for the removal of Iodine stains would tend to cause the neutralization of these chemistries to concentrations that would render them virtually to trace amounts. Thus the threat for an allergenic reaction is remote with either product.

One of the problems with the prior art formulations is the efficacy of the Iodine stain removal capacity. Sodium thiosulfate is less reactive than sodium metabisulfite when comparing its reducing action on Iodine stains. As can be appreciated, the use of sodium thiosulfate in an alkaline detergent type matrix may assist somewhat in general stain removal, however, the applicant has determined through actual usage that the effectiveness of this approach is weak as compared to modest aqueous solutions of sodium metabisulfite. It is believed that the neutralization of Iodine increases its solubility in any cleaning solution, therefore the emphasis in Iodine stain removal is preferably on the strength of the reducing agent. Once reduced, the Iodine is converted to a salt which is extremely soluble in water. In addition, the neutralization also eliminates the reactive characteristic, and therefore the irritant potential associated with prolonged contact of Iodine with the skin. Therefore, the prior art chemistry has not only been less effective in stain removal but it has also elevated the potential for leaving a greater concentration of residual Iodine that may result in skin irritation or other adverse reactions.

The prior art solutions utilizing alcohol also raise problems with respect to patient sensitivities. Many times a person leaving surgery and during recovery, feels cold and will immediately report of discomfort in this way. Staff will often respond with warm blankets that are available to ameliorate patient's discomfort. The application of a solution at this point, to remove Iodine stains, can exacerbate the patient's feeling of “cold” and discomfort. This is even more the case when solutions using an alcohol constituent are applied to the patient's skin, since the alcohol will quickly evaporate once applied which reduces skin temperature further and aggravates the patient's discomfort. Add to this the propensity for alcohol to absorb skin oils, the use of some of the prior art preparations can dry out the patient's skin resulting in aggravation of the incision or possibly leading to secondary conditions such as bedsores or infections.

The use of sodium metabisulfite in cleaning preparations is known in applications relating to the removal of cosmetics. For instance, U.S. Pat. No. 6,500,441 (Bui-Bertrand, et al) discloses an aqueous cleaning solution for the removal of cosmetics around the eyes. The attribute of the sodium metabisulfite in this case is related to the enhanced of dissolution of parabens used in the cleaning solution. Sodium metabisulfite was shown to substantially improve the solubility of methyl parabens and related paraben compounds in an aqueous matrix, which helps to extend the shelf life of the product and maintain the solution in pristine condition for use. It is noteworthy that the observations of the inventors confirmed that the sodium metabisulfite did not contribute to any irritation or other adverse reactions in and about the eyes of users.

The long-standing problems associated with Iodine staining have been addressed by the present invention, and its different embodiments, all of which will be disclosed in more detail below.

### SUMMARY OF THE INVENTION

A preparation for the cleaning of Iodine stains comprises a solution of chemistry from the family of metabisulfites in water ranging in concentration from 0.01 to 0.04 percent by weight of metabisulfite to water. A preferred range of use and selection comprises a solution of sodium metabisulfite in water ranging in concentration from 0.02 percent to 0.035 percent by weight, metabisulfite to water.

An Iodine stain removal solution of the present invention can be packaged as individual towelettes or wipes for use by medical staff or first aid technicians in removing Iodine stains from patients.

In an alternate version of the present invention, a solution of sodium metabisulfite, in water, in an effective range of concentration also includes a surface-active agent for improved wetting of the solution.

These and other attributes of the present invention will be discussed in more detail below.

### BRIEF DESCRIPTION OF THE DRAWINGS

FIG. 1 is an isometric view of a dispensing box of packaged Iodine stain removal wipes of the present invention.

FIG. 2 is an isometric view of an individual package of an Iodine stain removal wipe of the present invention.

FIG. 3 is an isometric view of the individual package of an Iodine stain removal wipe of FIG. 2, shown with a tear-off portion that exposes the wipe in the ready-to-use condition.

### DETAILED DESCRIPTION OF THE PREFERRED EMBODIMENTS

The present invention relates to the novel use of metabisulfites for the removal of Iodine stains in clinical or first aid situations. As mentioned above, the usage of Iodine related products is very predominant in health care, both human and veterinary, and the most common uses are found in the preparation of patients for surgical procedures and also in the cleansing of wounds as a first aid technique. Even with the advent of specialized Iodine products such as Betadine and Duraprep, the long-standing issues of staining persist. The staining issues relate not only to those of Iodine on the skin, but also Iodine stains on clothing, linens, flooring, and the like. This is a situation so prevalent that it forms a part of the daily considerations of those engaged in the health care systems, both human and veterinary.

The need for the present invention arose directly from the applicant's efforts to deal with the staining problems on an occupational basis. The persistence of Iodine on the skin of patients acts as an irritant as well as being unsightly. Concerns for the welfare of the patient meant that the efficacious removal of the Iodine was necessary to ensure that the patient's post-surgical experience was as comfortable and as uneventful as possible. Resort to the available products for this purpose was not satisfactory given the deficiencies described above. It was realized that the essence of Iodine stain removal was in the neutralization of Iodine, i.e., the reduction of the Iodine from an oxidated state. The reducing potential for sodium metabisulfite, as compared to sodium thiosulfate for instance, is greater thus making it more effective chemistry for neutralizing Iodine stains. The circumstance that led to an effective resolution forms the basis for the present invention, which has proven to be more effective in use than any other product to date.

Various ways of practicing the invention can be conceived without departing from the novel aspects of using metabisulfites for the removal of Iodine staining. The initial consideration is the makeup of solution with an effective concentration of the select metabisulfite. The method of delivering this solution to the subject area, whether this is the skin of a patient, or on clothing, or otherwise, is another consideration.

Metabisulfite Solution

Metabisulfite chemistry is typically acquired as an alkali or metal salt. The most common form, by far, is sodium metabisulfite, although it is believed that other members from the group of metabisulfites would also perform adequately so long as they exhibit the following characteristics: they are soluble in water, they have the ability to reduce (neutralize) Iodine from an oxidated state, they are relatively non-toxic and they do not provoke irritation or allergic reactions. In the present case, sodium metabisulfite is an excellent selection for this purpose since it meets the foregoing criteria and can be purchased in commercial quality and quantities. In addition, sodium metabisulfite, in the concentrations under consideration for the present invention, is extremely safe for use and is non-toxic. Additionally, it is believed that it possesses stability in aqueous solution. For the purposes of the following discussion it will be assumed that sodium metabisulfite is the chemical of choice although it is understood that one skilled in the art could substitute other metabisulfites in its place with corresponding adjustment of concentrations.

The makeup of effective solutions of the Iodine remover of the present invention is simply a matter of dissolving the sodium metabisulfite in water, preferably distilled water. From trial and error testing it has been determined that effective ranges of concentration run from 0.01 to 0.04 percent by weight, sodium metabisulfite to water. In use, the desired level of concentration may be intentionally varied to correspond to anticipated situations. For instance, products that are directed towards general cleanup, such as flooring, or equipment, or linens, may be made at the higher ranges so as to ensure quick and complete neutralization of the Iodine. The higher concentrations serve also to overcome any associated soil or other materials that may impede the action of the sodium metabisulfite on the Iodine stains. Higher concentrations than those described may be used without defeating the effects of sodium metabisulfite as a reducing agent and its capability in neutralizing Iodine stains, however, the use of sodium metabisulfite above the recommended levels increases the potential for allergenic responses when the intention is to use the product on human or animal patients, and represents an economic waste in terms of the core effectiveness gained.

With respect to the use of the metabisulfite solution of the present invention directly on human and animal patients, a specific range for effective concentrations has been shown to be 0.020 to 0.035 percent by weight of sodium metabisulfite to water. At these levels, the sodium metabisulfite is highly effective in eliminating Iodine staining and does not show any deleterious effects or irritation to the patient. Additionally, the same solution is effective for removing stains elsewhere. The solutions of the present embodiments may be packaged for liquid application, such as in bottles, or sprays, or swabs.

The metabisulfite solution of the present invention may also include other chemistry to enhance its applications. For instance, the inclusion of wetting agents would facilitate the metabisulfite contact with Iodine staining by increasing the ability of the solution to “wet out” the affected area, allowing it to penetrate the stain to a degree beyond that of a plain aqueous solution without the wetting agent. Wetting agents are known that are biodegradable and water soluble in neutral pH conditions. Given that sodium metabisulfite is sensitive to alkaline environments, the use of a select wetting agent would impart some detergent-like qualities without unnecessarily elevating the pH of the solution. The actual selection of a wetting agent is well within the ability of one skilled in the art and requires selecting one that is compatible with the pH and concentration of the preferred metabisulfite solution. It is believed that other enhancements of the preferred metabisulfite solution can be conceived without departing from the spirit and scope of the teachings herein.

Metabisulfite Wipes

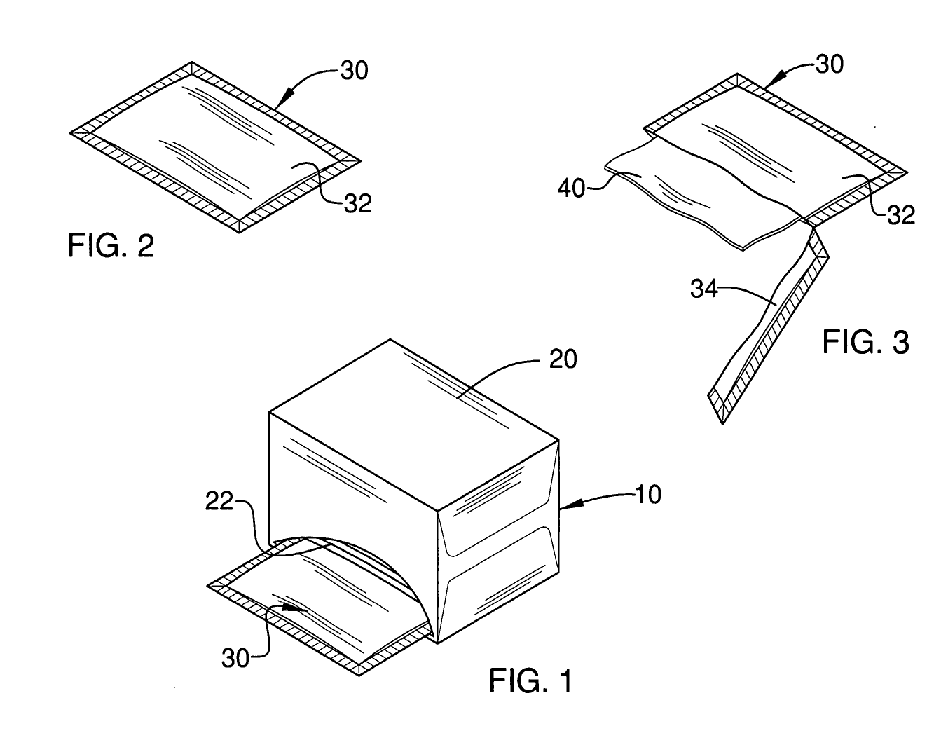

A metabisulfite wipe of the present invention may be seen in the drawings, where in FIG. 1, a box 10 is shown with individual wipe packages 30. The box 10 may be configured as a dispenser 20 with dispenser opening 22. As can be seen in FIGS. 2 and 3, the individual wipe packages 30 can be supplied with outer wrap 32 and with tear off portion 34. The actual metabisulfite saturated wipe 40 is revealed in FIG. 3.

The wipes of the present embodiment would be directed towards clinical environments where health care staff can use a convenient means to clean Iodine stains from a patient or an Iodine stained surface in the clinical environment. As may be surmised from the drawings, a wipe of the present invention can be enclosed in compatible packages, similar to other cleaning wipes used in restaurants and the like, where the wipes can be accessed on an as-needed basis. The actual wipe is comprised of a pad that is typically folded to fit within the package, and it is saturated with the metabisulfite solution of the present invention. The wipes are kept in a saturated condition while in the package, the package itself being impermeable thereby preventing the wipe from drying out. When used, the package is opened by tearing along one edge, exposing the wipe inside, which is removed from the package and thereafter it may be used “as is” to wipe Iodine stains, or it may be unfolded to handle larger areas of staining. Once it has been used, it will contain some amount of neutralized Iodine which is not considered to be an environmental threat, however it may be considered a biohazard and would be disposed in accordance with the procedures for handling biohazards. The wipes are preferably intended to be disposed of after one use.

While the wipes may be used as they come out of the package, it is also possible to store packages in a device such as a blanket warmer. Blanket warmers are commonly found in the clinical environment and in particular are used to aid patients in post-surgical situations. The warmth of the blanket adds comfort since many patients experience severe chilling during the surgical procedure and especially during the recovery stage, anything that can be done to facilitate the comfort of the patient is deemed to be a positive. The packages of the present invention, with the wipe containing the metabisulfite solution, can similarly be placed in the blanket warmer and elevated to a temperature that would minimize the chilling effect that one might ordinarily feel when a moist tissue is applied to a person's exposed skin. Additionally, the effect of elevating the temperature of the wipe will increase its ability to clean the Iodine staining, since reactivity varies proportionally with increases in temperature.

The warming effect on the wipes of the present invention can also be beneficially applied when the patient is an infant. The prior art solutions for Iodine neutralization typically contain alcohol which only exacerbates the chilling effects of using a moist, room temperature wipe. The wipes of the present invention, especially when pre-warmed, can greatly facilitate the cleaning of Iodine stains on infants.

Veterinary Metabisulfite Solutions

The usage of the prior art Iodine removers in veterinary applications poses a specific set of problems. The application of products containing alcohol have the same effect as mentioned above with the chilling of patients. In veterinary situations, the “patient” cannot easily be comforted as is the case when dealing with human patients. Thus the avoidance of alcohol chemistry is a substantial benefit over that of the prior art chemistry. In addition, many time the Betadine swabbing extends into areas on the patient beyond the subject incision area. Iodine staining is a more complex problem in this instance since the hair of the dog or cat, for instance, wicks the Iodine chemistry deep into the coat and will cause irritation, and even burning, if not completely neutralized. The metabisulfite solutions of the present invention, being more effective chemically, reduce the chances of irritation and burning in these situations.

The use of a metabisulfite solution with a wetting agent further enhances the ability of the chemistry to react with the Iodine residues, since it will penetrate the coat more thoroughly. This has the effect of washing the coat of the pet out more completely and ensures that the metabisulfite chemistry has a chance to reach the stained areas.

In general, the use of a metabisulfite solution for Iodine stain removal is believed to represent a new approach to the long-standing problems encountered in the clinical and veterinary environments. The products and embodiments of the present invention may, in addition, find their way into the home environment as well, where the use of the tincture of Iodine still occurs at times. The benefits of the application of more effective reducing chemistry have been readily observed in actual testing of the present invention in real-world environments. It is believed, therefore, that the teachings herein are illustrative of the potentials for use of the present invention and do not represent limitations or restrictions.

I claim:

1. A preparation for the removal of Iodine chemical stains, the preparation comprising:
An aqueous matrix;
A selected metabisulfite dissolved in said aqueous matrix;
Where said metabisulfite is capable of neutralizing Iodine chemical stains when the preparation is applied to such stains.

2. The preparation for the removal of Iodine chemical stains of claim Number 1, where the selected metabisulfite is sodium metabisulfite.

3. The preparation for the removal of Iodine chemical stains of claim Number 2, where the concentration of the sodium metabisulfite is in the range of 0.01 to 0.04 percent by weight, sodium metabisulfite to water.

4. The preparation for the removal of Iodine chemical stains of claim Number 3, where the preferred concentration of sodium metabisulfite is in the range of 0.02 to 0.035 percent by weight, sodium metabisulfite to water.

5. The preparation for the removal of Iodine chemical stains of claim Number 1, where the preparation further includes a wetting agent.

6. A towelette for removing Iodine chemical stains, the towelette comprising:
A wipe saturated with a selected metabisulfite in aqueous solution; and,
A package for enclosing and protecting said wipe until it is to be used.

7. The towelette of claim Number 6, where the metabisulfite is sodium metabisulfite.

8. The towelette of claim Number 7, where the sodium metabisulfite concentration is in the range of 0.01 to 0.04 percent by weight, sodium metabisulfite to water.

9. The towelette of claim Number 8, where the preferred range of sodium metabisulfite concentration is 0.02 to 0.035 percent by weight, sodium metabisulfite to water.

10. The towelette of claim Number 8, where the towelette and the aqueous metabisulfite solution can be warmed before use.

11. The towelette of claim Number 6, where the aqueous metabisulfite solution further includes a wetting agent.

12. A method for using an aqueous solution of a metabisulfite to remove Iodine chemical stains, the steps of which comprise:
Selecting a packaged wipe that is saturated with an aqueous solution of metabisulfite;
Opening the package;
Extracting the wipe from within the package;
Applying the wipe to an Iodine chemical stain;
Disposing of the soiled wipe in an appropriate manner

13. The method for using an aqueous solution of a metabisulfite to remove Iodine chemical stains of claim Number 12, where the aqueous solution of metabisulfite is a sodium metabisulfite solution with the concentration of sodium metabisulfite in the range of 0.01 to 0.04 percent by weight, sodium metabisulfite to water.

14. The method for using an aqueous solution of a metabisulfite to remove Iodine chemical stains of claim Number 12, where the method further includes the step of warming the wipe prior to applying the wipe to an Iodine chemical stain.
